(19) United States
(12) Patent Application Publication
(10) Pub. No.: US 2011/0045498 A1
(43) Pub. Date: Feb. 24, 2011

(54) On-Site Liquid Production

(76) Inventors: Martin Lindberg, Malov (DK); Lars Winther, Smorum (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/675,232

(22) PCT Filed: Aug. 25, 2008
(86) PCT No.: PCT/DK2008/000300

§ 371 Date: Oct. 28, 2010

Related U.S. Application Data
(60) Provisional application No. 60/966,604, filed on Aug. 29, 2007.

(51) Int. Cl. G01N 33/567; C12M 1/02; C12M 1/36

(52) U.S. Cl. 435/7.21; 435/283.1; 435/286.1

(57) ABSTRACT

A biological sample processing system for on-site liquid production comprises a processing apparatus for processing of biological samples arranged on microscope slides, and a production unit connected to the apparatus. The production unit comprises a first ingredient source, a second ingredient source, and a mixer station. The mixer station is configured to mix ingredients to produce a liquid product. Supply conduits are arranged to supply an amount of a first ingredient and an amount of a second ingredient from the first and second ingredient sources to the mixer station. A delivery conduit is provided for transportation of an amount of the liquid product from the mixer station. A production controller is arranged in communication with the production unit, and is configured to control the operation of the production unit.

### TECHNICAL FIELD

Embodiments of the present invention relate to systems and methods for on-site liquid production, and especially to biological sample processing systems and methods for on-site liquid production. Embodiments relate further to biological sample processing systems and methods for automatic and/or on demand on-site liquid production.

### BACKGROUND

In biological sample processing systems of today, a need exists for automatic and continuous processing of biological samples. The main reasons being that an increasing number of biological samples are to be processed, e.g., by a hospitals' pathology laboratory, in as short time as possible. At the same time, the number of qualified personnel working in the laboratories is decreasing. Thus, a reduced number of people are to process a larger number of biological samples in a reduced time period.

In order to speed up and to facilitate the biological sample processing, automated sample processing systems, e.g. automated sample staining systems, exist. In this disclosure, the term “staining” is used to refer to the end product of a process, by which certain parts of the sample may be stained, i.e. have obtained a different color, either in the optic range or in another electromagnetic range, such as ultra violet, or the staining may be a detectable (e.g., automatically detectable) change in properties, such as fluorescent properties, magnetic properties, electrical properties or radioactive properties. To obtain the staining, the sample may undergo a series of treatment steps, such as—but not limited to—washing, binding of reagents to the specific parts of the sample, application of the reagents, etc. and each treatment step may include a plurality of individual treatments.

Examples of sample preparation and processing that may be used in the practice of the invention include but are not limited to the following.

Sample processing in immunohistochemical (IHC) applications and in other chemical and biological analyses may involve one or a number of various processing sequences or protocols as part of an analysis of one or more samples. The sample processing sequences or protocols may be defined by the individual or organization requesting an analysis, such as a pathologist or histologist of a hospital, and may be further defined by the dictates of a particular analysis to be performed.

In preparation for sample analysis, a biological sample may be acquired by known sample acquisition techniques and may comprise, for example, in IHC applications, tissues generally or, even, in some applications one or a plurality of isolated cells, such as in microarray samples, and may be presented on a microscope slide or a similar plane, sample carrier. Furthermore, the sample may be presented on the slide or other carrier variously and potentially in some form of preservation. As one example, a sample such as a layer or slice of skin may be preserved in formaldehyde and presented on a slide with one or more paraffin or other chemical layers overlying the sample. Samples preserved with paraffin may need to undergo deparaffinization, a process by which paraffin layers overlaying and/or infiltrating the sample are removed. In addition, the target or sample may need to be restored to a condition where it is suitable for staining operations—a process known as target retrieval.

Immunologic applications, for example, may involve processing sequences or protocols that comprise steps such as deparaffinization, target retrieval, and staining, especially for in-situ hybridization (ISH) techniques.

The staining procedure may be laborious and use many different kind of liquids, e.g. reagents. The staining protocol may include the following steps: deparaffinization, washing, antigen retrieval, endogenous biotin or enzyme blocking, incubation with immunological reagents, molecular probes, secondary visualization reagents and various chromogen reagents, washing steps and counterstaining.

Some liquids, sometimes called bulk fluids, used in the sample processing are used so frequently and in such large volumes that they often are provided in containers of large volume in order to save the personnel from needing to frequently replenish those containers. Examples of such liquids are washing solutions, buffer solutions, deparaffinization solutions, target retrieval liquids, ISH stringency wash buffers, etc. The volumes of the containers are often in the range of 2 to 15 liters. However, even if the containers do not have to be replaced so often it may nevertheless be heavy and cumbersome for the personnel to handle a new full container and to put it in its position.

Further, many laboratories of today have to fit many more instruments than previously within the same laboratory space, whereby the demands on effective utilization of the footprint of each instrument is increased. It is therefore also a need to reduce the number of space demanding liquid containers within the laboratory.

Furthermore, some sample processing liquids, e.g. some reagents, have short shelf lives and may be used so infrequently and/or in so small volumes that their quality might deteriorate due to the short shelf life before all the liquid has been used. It is therefore also a need to produce liquids on demand in order to reduce the amount of sample processing liquids to be thrown away due to expired expiration dates.

Currently available biological sample processing system do not provide for on-site liquid production. Especially, currently available biological sample processing systems do not provide for on-site liquid production in an automatic and/or on demand fashion.

In this disclosure the term “on-site” is used to refer to a location in proximity to the sample processing system, and preferably to a location in close proximity to the sample processing system. For example, the location could be within the same room of a building as the sample processing system is located or it could be a location within the same laboratory as the sample processing system is located.

Before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. For example, not all elements of the embodiments described are required to achieve at least one of the advantages described herein. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### SUMMARY OF THE INVENTION

The present invention relate to systems and methods for on-site liquid production, and especially to biological sample processing systems and methods for liquid production. Embodiments of the invention relate to biological sample processing systems and methods for automatic and/or on demand on-site liquid production.

By on-site liquid production it is possible to produce liquid products in proximity to the biological sample processing site and to keep track of information in relation to the liquid production. For example, it may be possible to keep track of the ingredient sources that are used for producing the liquid products, e.g. identification labels of the ingredient sources, the content of the ingredient sources, where the ingredient sources are located in the production unit, when they were opened, when they will expire, the volume of the ingredient remaining in an ingredient source, etc. Further, it is possible to keep track of the liquid products produced, e.g. identification labels of the liquid product containers, the content of the liquid products, when the liquid products were produced, when they will expire, the volume of the liquid product, the volume of the liquid product remaining in the liquid product container, where the liquid product containers are located, etc.

Embodiments of the invention relates to a biological sample processing system, comprising a biological sample processing apparatus for processing of biological samples arranged on microscope slides and a production unit connected to the biological sample processing apparatus. The production unit comprises a first ingredient source of a first ingredient; a second ingredient source of a second ingredient; a mixer station comprising a mixing chamber having a mixer inlet and a mixer outlet, the mixer station being configured to mix ingredients in the mixing chamber to produce a liquid product; a first supply conduit for supply of an amount of the first ingredient from the first ingredient source to the mixing chamber via the mixer inlet; a second supply conduit for supply of an amount of the second ingredient from the second ingredient source to the mixing chamber via the mixer inlet; and a delivery conduit for transportation of an amount of the liquid product from the mixing chamber via the mixer outlet. The sample processing system comprises further a production controller arranged in communication with the production unit. The production controller comprises a processor; and a memory device in communication with the processor, the memory device comprising a computer program which the processor is configured to access to control the operation of the production unit.

In embodiments, the biological sample processing system comprises further a sample processing apparatus controller SC arranged in communication with the sample processing apparatus, and being configured to control and monitor various processes, functions, or components, that are implemented on or relate to the operation of the sample processing apparatus.

In embodiment, the sample processing apparatus controller SC is arranged to schedule the production of liquids in the production unit. The sample processing apparatus controller SC may be arranged to either directly or indirectly schedule the production of liquid products in the production unit.

Embodiments comprise further a system manager server SMS arranged in communication with the production controller and the sample processing apparatus controller SC, and configured to control the production controller, and wherein the sample processing apparatus controller SC is configured to transfer data to and from the system manager server SMS.

Embodiments comprise further a system manager SM arranged in communication with the system manager server SMS and configured to provide a user to connect to, and to transfer data to and from the system manager server SMS.

In embodiments the mixer inlet is provided with a spray nozzle configured to supply an amount of the ingredient to the mixing chamber in a spray pattern. The mixer station comprises a mixing means configured to mix the ingredients in the mixing chamber upon rotation.

In embodiments, the second ingredient source comprises an internal screw controlled by the production controller and configured to meter a predetermined amount of the second ingredient for supply to the mixer station.

Embodiments comprise further a water tap, water conduit and a water supply regulator for supplying an amount of water to the first ingredient source or to the mixer station. A purifier may be arranged to purify the water supplied from the water tap. The purifier may be a filtration device or a separator configured to use reverse osmosis to purify the water.

It should be understood that in embodiments where water is supplied from the water tap to the mixer station, the water tap could be considered as the first ingredient source and the water as the first ingredient.

Embodiments comprise further at least one quality control station arranged to check the quality of liquid supplied to, supplied within or supplied from the production unit.

In embodiments, the biological sample processing system comprises further a first supply flow regulator arranged at the first supply conduit, arranged to be controlled by the production controller and arranged to supply an amount of the first ingredient to the mixing chamber in response to control instructions from the production controller; a second supply flow regulator arranged at the second supply conduit, arranged to be controlled by the production controller and arranged to supply an amount of the second ingredient to the mixing chamber in response to control instructions from the production controller; and a delivery flow regulator arranged at the delivery conduit, arranged to be controlled by the production controller and arranged to transport an amount of the liquid product from the mixing chamber in response to control instructions from the production controller.

In embodiments the delivery flow regulator is configured to transport an amount of the liquid product to a second or third quality control station arranged to control the quality of the liquid product, and configured to transport quality controlled liquid product to a production unit outlet or to a liquid storage station for storage.

In embodiments, the liquid storage station comprises a storage chamber having a storage inlet connected, via storage inlet conduit, to the third quality control station and the delivery flow regulator; and a storage outlet connected, via storage outlet conduit, to a fourth quality control station and to a production unit outlet.

Embodiments may further comprise an antimicrobial device arranged in the liquid storage station and configured to prevent bacterial growth in the liquid storage station, and wherein the antimicrobial device comprises a heat source or a UV source.

In embodiments the production unit outlet is connected to a liquid product tap by means of which an amount of the liquid product can be withdrawn from the production unit.

Embodiments comprise further one or more fluid containers arranged at one or more fluidics carts, the one or more fluid containers being in fluid communication with the production unit outlet, whereby liquid product can be supplied from the production unit to one or more of the fluid containers. The fluidics cart may be arranged in communication with the sample processing apparatus controller SC that is configured to control the supply of fluid from the fluid container on the fluidics cart to the sample processing apparatus.

Embodiments may further comprise a weighing means arranged at the mixer station and arranged in communication with the production controller, the weighing means is configured to weigh the mixing chamber to determine the amount of ingredient comprised in the mixing chamber and configured to communicate the amount of ingredient to the production controller, whereby the production controller can determine if more ingredient is to be supplied or if sufficient ingredient has been supplied and whereby the production unit controller can control the production unit accordingly, e.g. to supply more ingredient or to stop supplying ingredient.

Embodiments may further comprise at least one level sensing mechanism arranged in the mixer station and arranged in communication with the production controller, the level sensing mechanism being configured to sense the filling level in the mixing chamber and configured to communicate the filling level to the production controller, whereby the production unit controller can control the production unit in dependence of the filling level.

In embodiments, the first ingredient source and second ingredient source are disposable and/or collapsible containers. At least one of the first and second ingredient sources may be configured as a flexible bag comprising the first or second ingredient, and wherein the flexible bag is comprised in a box in a so-called bag-in-a-box configuration.

In embodiments, the liquid product is produced as a batch or is produced continuously. The liquid product may be produced on demand. The liquid product may be automatically produced.

In embodiments, the first ingredient is purified water, the second ingredient is a buffer concentrate and the liquid product is a buffer solution.

In embodiments, the production controller is configured to control the production unit to produce the liquid product in dependence on the consumption of the liquid product by the sample processing apparatus.

Embodiments of the invention relate to a method for liquid production in a biological sample processing system. The method comprises the steps of:
providing a biological sample processing apparatus for processing of biological samples arranged on microscope slides;
providing a production unit connected to the biological sample processing apparatus, the production unit comprising:
   a first ingredient source of a first ingredient;
   a second ingredient source of a second ingredient;
   a mixer station comprising a mixing chamber having a mixer inlet and a mixer outlet, the mixer station being configured to mix ingredients in the mixing chamber to produce a liquid product;
providing a production controller arranged in communication with the production unit, the production controller comprising:
   a processor; and
   a memory device in communication with the processor, the memory device comprising a computer program which the processor is configured to access to control the operation of the production unit;
supplying an amount of the first ingredient from the first ingredient source to the mixing chamber in dependence of control instructions from the production controller;
supplying an amount of the second ingredient from the second ingredient source to the mixing chamber in dependence of control instructions from the production controller;
mixing in the mixer chamber the amount of the first ingredient and the amount second ingredient in dependence of control instructions from the production controller to produce the liquid product, and
transporting an amount of the liquid product from the mixing chamber via the mixer outlet.

Embodiments comprise further the steps of: providing a sample processing apparatus controller SC in communication with the sample processing apparatus, and controlling and monitoring various processes, functions, or components, that are implemented on or relates to the operation of the sample processing apparatus by means of the sample processing apparatus controller SC.

Embodiments comprise further the step of scheduling the production of liquids in the production unit by means of the sample processing apparatus controller SC. The production of liquids in the production unit may be either directly or indirectly scheduled by means of the sample processing apparatus controller SC.

Embodiments comprise further the steps of: providing a system manager server SMS arranged in communication with the production controller and the sample processing apparatus controller SC; and controlling the production controller by means of the system manager server SMS.

Embodiments comprise the steps of: providing a system manager SM arranged in communication with the system manager server SMS; and providing a user to connect to, and to transfer data to and from the system manager server SMS by means of the system manager SM.

In embodiments, the step of supplying an amount of the ingredients to the mixing chamber comprises the step of supplying an amount of the ingredient to the mixing chamber in a spray pattern.

Embodiments comprise the step of providing at least one quality control station arranged to check the quality of liquid supplied to, supplied within or supplied from the production unit.

Embodiments comprise the steps of providing a liquid storage station; and transporting an amount of the liquid product from the mixing chamber to the liquid storage station for storage.

Embodiments comprise the steps of: providing a production unit outlet connected to a liquid product tap; transporting an amount of the liquid product from the mixing chamber to the production unit outlet; and withdrawing an amount of the liquid product from the production unit by means of the liquid production tap.

Embodiments comprise the steps of: providing a production unit outlet connected to one or more fluid containers, and supplying an amount of the liquid product from the production unit to one or more of the fluid containers.

Embodiments comprise the step of producing the liquid product as a batch or continuously.

Embodiments comprise the step of producing the liquid product on demand.

Embodiments comprise the step of controlling the production unit to produce the liquid product in dependence on the consumption of the liquid product by the sample processing apparatus.

Embodiments of the invention relate to a computer-readable medium that stores computer program parts, which when executed by a processor controls steps in a method according to any of claim 16-24 or realizes features in a system according to any of claim 1-15.

### BRIEF DESCRIPTION OF THE DRAWINGS

The accompanying drawings, which are incorporated in and constitute a part of this specification, illustrate a number of non-limiting embodiments of the invention and together with the description, serve to explain the principles of the invention.

FIG. 1 schematically illustrates an embodiment of a biological sample processing system;

FIG. 2 is a schematic overview of an embodiment of a continuous workflow heterogeneous biological sample processing network;

FIG. 3 schematically illustrates an embodiment of a continuous workflow heterogeneous biological sample processing network;

FIG. 4 schematically illustrates a first embodiment of a mixer station;

FIG. 5 schematically illustrates a second embodiment of a mixer station;

FIG. 6 schematically illustrates a third embodiment of a mixer station;

FIG. 7 schematically illustrates an embodiment of an ingredient source for a powder ingredient; and

FIG. 8 schematically illustrates an alternative embodiment of a biological sample processing system.

### DETAILED DESCRIPTION OF THE INVENTION

Embodiments of present invention relate to systems and methods for on-site liquid production, and especially to biological sample processing systems and methods for on-site liquid production. Embodiments relate further to systems and methods for automatic and/or on demand on-site liquid production. Various modifications to the embodiments will be readily apparent to those skilled in the art and generic principles disclosed herein may be applied to other embodiments. The described examples are exemplary and embodiments should not be construed to limit the present invention to the systems, techniques, and applications explicitly described herein. The present invention is not intended to be limited to the embodiments shown but is to be accorded the widest scope consistent with the principles and features described herein.

The present disclosure relates, in part, to the field of software and hardware for the control, management, tracking, monitoring, scheduling and diagnosing of or in relation to liquid production, and especially in relation to liquid production in a biological sample processing system.

The term “comprise/comprising” when used in this specification is taken to specify the presence of stated features, integers, steps or components but does not preclude the presence or addition of one or more other features, integers, steps components or groups thereof.

In the figures, the same reference numerals are used for the same or similar components, features, steps or the like.

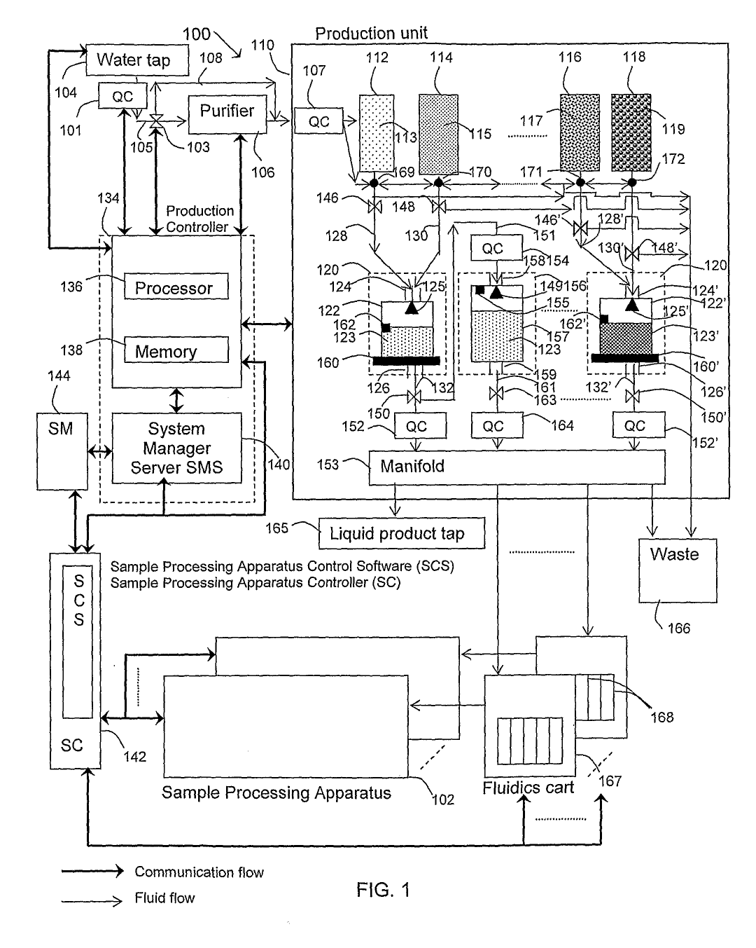

FIG. 1 shows an embodiment of a biological sample processing system 100 according to the present invention. In a preferred embodiment, the biological sample processing system 100 is an automated biological sample processing system.

In embodiments, the biological sample processing system 100 comprises at least one biological sample processing apparatus 102 for processing biological samples arranged on microscope slides. The biological sample processing apparatus may for example be a staining apparatus and especially an automated staining apparatus.

The Autostainer™ System (LabVision Corporation) is one example of an automated staining system. The stainer is compatible with currently available reagents for staining paraffin-embedded and frozen tissue sections, cytospins, cell smears, and fine-needle aspirates, for example. The stainer is designed to automate manual staining methods routinely used in immunohistochemistry and cytochemistry. The stainer has fluidic cart for containers comprising fluids, e.g. bulk fluids and fluidic wastes. Flexible programming allows for an unlimited number of protocols containing up to 35 steps, including rinse and blow steps between different processing steps, and 64 different reagents. A staining run can process from 1 to 48 microscope slides. Individual slides can be programmed to receive different reagents, of specified volume, during any step in a staining protocol, and waste is segregated into hazardous and non-hazardous collection containers, reducing disposal costs. The stainer is further designed to track a variety of data. It can generate patient, reagent, and real-time operation data reports, as well as track reagent usage and log instrument maintenance.

Embodiments comprise a system and method for the control of fluidic sub-system of the sample processing apparatus, wherein different fluids are supplied to baths or chambers in the sample processing apparatus within which slides may be processed in accordance with a protocol. Systems and methods provide a means to control the sub-systems to ensure the availability and flushing of the fluids in accordance with the protocol.

Some embodiments of the invention include a scheduler, in some embodiments referred to as a sample processing control system or sample processing apparatus controller, such that one or more groups of samples may be processed according to one or more protocols that may be automatically identified by the scheduler. In one embodiment, the scheduler(s) may be non-deterministic and adaptive, i.e. does whatever is next and whatever is next may change for a variety of conditions. In some embodiments, the scheduler may be configured to schedule the on-site production of liquid(-s) in dependence of the required liquid(-s) for the sample processing as may be defined in the sample processing protocols. As used herein tasks refer to steps that are executed according to a protocol. In some embodiments, sample groups or individual slides may be inserted or removed during processing protocol steps by the control and monitoring accomplished by the scheduler. In some embodiments, protocols may be indicated by information on the slides. Some embodiments of the present invention also include a system and method for defining new protocols that may be applied to carriers. Protocols may be defined or adapted at the apparatus or remotely through the internet or a network. Some embodiments of the invention include a system for the detection of incompatible or inconsistent protocols, and a system and method to prevent the use of incompatible reagents within a protocol or adaptation of protocols in view of mechanical failure such as temperature control failure or fluid failure such as lack of at least one liquid, e.g. reagent.

In embodiments, the biological sample processing system 100 comprises a production unit 110 connected to the biological sample processing apparatus 102. The production unit 110 may comprise a first ingredient source 112 for a first ingredient 113, a second ingredient source 114 for a second ingredient 115, and a mixer station 120. The mixer station 120 may comprise a mixing chamber 122 having a mixer inlet 124 and a mixer outlet 126. The mixer station 120 is configured to mix ingredients in the mixing chamber 122 to produce a liquid product 123.

It should be understood that more than two ingredient sources may be comprised in the system, and that the number of ingredient sources may depend on the different liquid products to be produced and on the number of different liquid products to be produced. It should also be understood that the ingredient source comprising an ingredient may be prefabricated or that the ingredient source may be delivered empty for later filling of an ingredient. Further, it should be understood that the production unit may be connected to an ingredient source, i.e. the ingredient source may be e.g. a water tap connected to the production unit by means of conduits.

An ingredient source is any medium that supports or provides an ingredient. For example, as used herein, an ingredient source includes a container, such as a bottle or vial that can hold at least one ingredient. It also includes a support for at least one container, such as a container rack. Ingredient source may further refer to a larger scale support, such as a rack holder, that holds at least one smaller support, such as a plurality of racks, each rack containing a plurality of containers. An ingredient source may releasable hold, securely hold, and/or hold in such a way that permits movement, such as vertical, horizontal or pivoting about one or more axis. In one embodiment, the ingredient source may function as an ingredient holding means. Alternative embodiments of a ingredient source comprise one or more carousels, trays, racks, carriers, holders, compartments, or other conveyance arrangements used for the handling and processing of ingredients and ingredient carriers any of which may be at least partially removable.

In one embodiment, the system may include means for monitoring and receiving, viewing, inputting, programming, analyzing, and editing data. For example, some embodiments of the invention provide a Graphical User Interface (GUI) to allow user input and control of the production unit and/or the sample processing apparatus. Other embodiments include an integrated touch screen, remote clients including workstations, PCs, internet, cell phones, PDAs, and pagers. Other embodiments include monitors within network connected or remotely connected instruments. Some embodiments of the invention provide a remote monitoring system that allows remote tracking and monitoring of the production unit and/or the sample processing apparatus including retrieval of diagnostic information about the production unit and/or the sample processing apparatus. Some embodiments of the present invention relate to a system and method for tracking liquid usage and ingredient usage, and system statistics to make predictive determinations with regard to pre-ordering supplies.

As shown in FIG. 1, the production unit 110 may comprise an (L−1)^{th} ingredient source 116 for an (L−1)^{th} ingredient 117 and an L^{th} ingredient source 118 for an L^{th} ingredient 119, where L is an arbitrary integer greater than 1. Depending on the liquid products to be produced, additional mixer stations may be provided. FIG. 1 also shows an N^{th} mixer station 120′, where N is an arbitrary integer greater than 1.

The production unit 110 may further comprise supply conduits 128, 130, 128′, 130 for the supply of an amount of one or more ingredients 113, 115, 117, 119 from one or more of the ingredient sources 112, 114, 116, 118 to one or more of the mixer stations 120, 120′.

In this disclosure, the term “conduit” is used to refer to means for conveying matter from one location to another, e.g. from one component or part of the system to another, or from one place to another place. The conduit could be a tube, tubing, pipe, hose or the like. The conduit could also be a combination of one or more tubes, tubings, pipes, hoses or the like. The conduit could be of a flexible or rigid type.

As illustrated in FIG. 1, the production unit 110 may comprise a first supply conduit 128 for the supply of an amount of the first ingredient 113 from the first ingredient source 112 to the mixing chamber 122 via the mixer inlet 124. A second supply conduit 130 may be arranged for the supply of an amount of the second ingredient 115 from the second ingredient source 114 to the mixing chamber 122 via the mixer inlet 124. Further, a delivery conduit 132 may be arranged to transport an amount of the liquid product 123 from the mixing chamber 122 via the mixer outlet 126.

In embodiments, the (L−1)^{th} ingredient source 116 may be configured to supply an amount of the (L−1)^{th} ingredient 117 via the first supply conduit 128 or the second supply conduit 130 to the first mixer station 120, or via an (L−1)^{th} supply conduit 128′ to an N^{th} mixer station 120′ comprised in the production unit 110.

In embodiments, the L^{th} ingredient source 118 being configured to supply an amount of the L^{th} ingredient 119 via the first supply conduit 128 or the second supply conduit 130 to the first mixer station 120, or via an L^{th} supply conduit 128′ to the N^{th} mixer station 120′.

The N^{th} mixer station 120′ may have a design and/or function similar or almost similar to the design and/or function of the first mixer station 120. The N^{th} mixer station 120′ may comprise a mixing chamber 122′ having a mixer inlet 124′ and a mixer outlet 126′. The mixer station 120′ is configured to mix ingredients in the mixing chamber 122′ to produce a liquid product 123′.

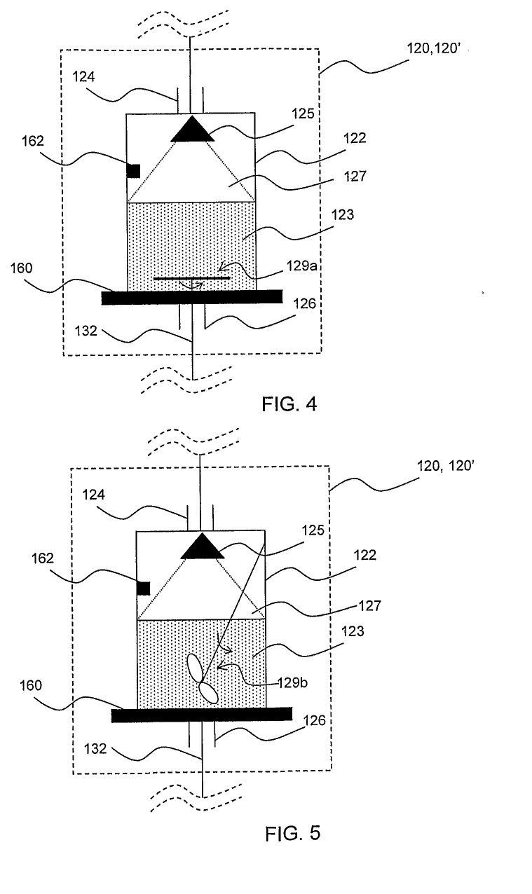

In embodiments, the mixer inlet 124, 124′ is provided with a spray nozzle 125, 125′ configured to supply an amount of the ingredient 113, 115, 117, 119 to the mixing chamber 122, 122′ in a spray pattern 127, 127′ and thereby improving the mixing efficiency, cf. FIGS. 4-6.

In embodiments, the mixer station 120, 120′ comprises mixing means 129, 129′. The mixer station 120, 120′ may be of a blender type comprising a blade assembly 129a, 129a′ in the bottom part of the mixer chamber 122, 122′, cf. FIG. 4. The blade assembly 129a, 129a′ is rotatably arranged within the mixing chamber 122, 122′ and configured to mix the ingredients in the mixing chamber 122, 122′ upon rotation.

In another embodiment, the mixer station 120, 120′ may be of a mixer type having an impeller or a stirrer 129b, 129b′ that is rotatably arranged within the mixing chamber 122, 122′ and configured to mix the ingredients in the mixing chamber 122, 122′ upon rotation, cf. FIG. 5. The impeller may be a paddle, pitched turbine, propeller or the like. Other mixing methods may include side entering jets of fluid, such as liquid. It should also be understood that inlet and outlet conduits from tanks may comprise motionless mixers for further homogenizing the liquids or paste.

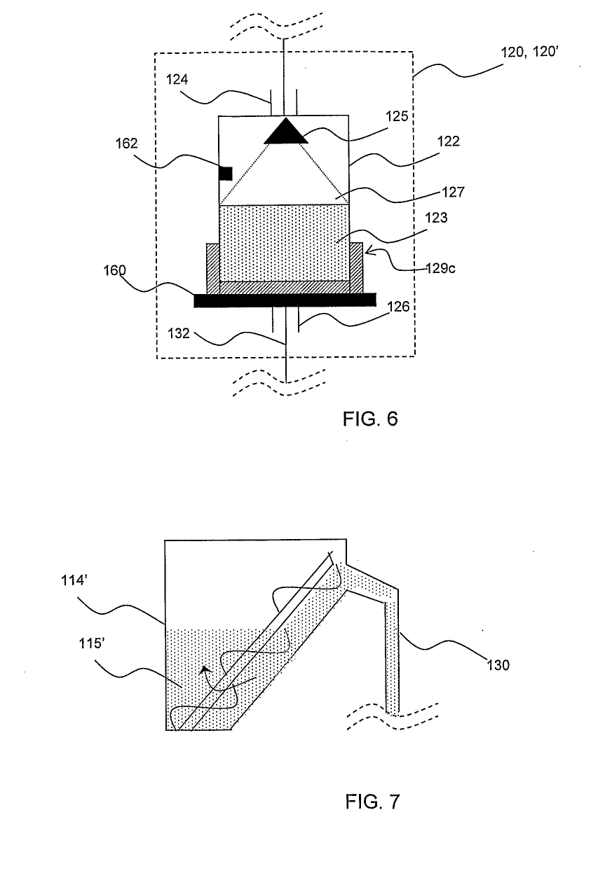

In yet another embodiment, the mixer station 120, 120′ comprises a vortex mixer 129c, 129c′ configured to mix the ingredients in the mixing chamber 122, 122′, cf. FIG. 6.

The biological sample processing system 100 comprises further a production controller 134 arranged in communication with the production unit 110. In embodiments, the production controller 134 may be comprised in the production unit 110.

The production controller 134 comprises a processor 136, and a memory device 138 in communication with the processor 136. The memory device 138 comprises or is configured to store a computer program or computer program parts, which the processor 136 is configured to access in order to control the operation of the production unit 110. The processor 136 may be a microprocessor and the memory device 138 may be a volatile memory such as a Random Access Memory (RAM) or a non-volatile memory such as a Read Only Memory (ROM).

In embodiments, the biological sample processing system 100 comprises further a system manager server SMS 140 communicatively connected to the production controller 134 and configured to control the production controller 134. The sample processing system 100 may further comprise a sample processing apparatus controller SC 142 arranged in communication with the sample processing apparatus 102 and the production controller 134. The sample processing apparatus controller SC 142 may be arranged in communication with the production controller 134 either directly or via the system manager server SMS 140. The sample processing apparatus controller SC 142 is configured to control and monitor various processes, functions, or components, etc. which are implemented on or relate to the operation or control of the sample processing apparatus 102. The sample processing apparatus controller SC 142 may be configured to transfer data to and from the production controller 134 and/or the system manager server SMS 140.

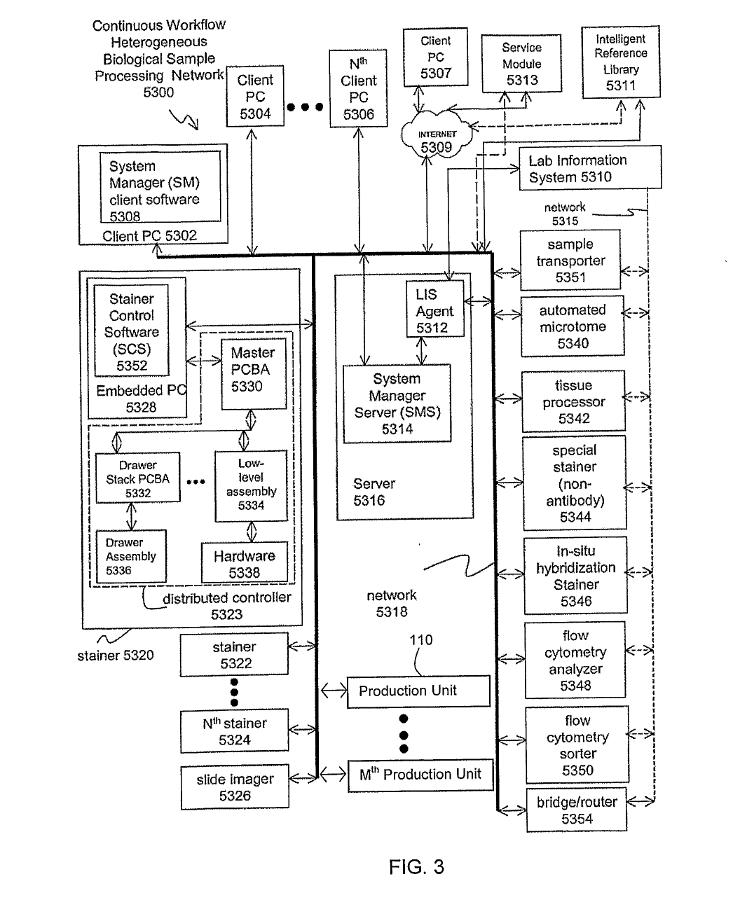

Embodiments may further comprise a system manager SM 144 arranged in communication with the system manager server SMS 140. The system manger SM 144 may be configured to provide a user to connect to, and to transfer data to and from the system manager server SMS 140. The system manager SM 144 may be comprised in or realized as a client PC 5302, cf. FIG. 3.

In embodiments, the production controller 134 comprises the system manager server SMS 140 as illustrated by the dotted line in FIG. 1.

In embodiments, the first ingredient source 112 may be a water source and the first ingredient 113 may be water. The water may be purified water, such as filtered water or ion-exchanged water.

In embodiments, the second ingredient 115, the (L−1)^{th} ingredient 117 and/or the L^{th} ingredient 119 is to be diluted by or dissolved in the first ingredient 113.

In embodiments, one of the ingredients 113, 115, 117, 119, e.g. the first ingredient 113, is purified water. Further, the second ingredient 115, the (L−1)^{th} ingredient 117, and/or the L^{th} ingredient 119 may comprise:

a) a salt such as natrium chloride, Tris (trishydroxymethylaminomethane), litium chloride or phosphate;

b) a pH controlling substance such as hydrogen chloride, Tris, citrate, sodium hydroxide, sulphuric acid, phosphate, HEPES (4-(2-hydroxyethyl)-1-piperazineethane-sulfonic acid);

c) a metal chelate such as EDTA (ethylenediamine tetraacetic acid) and citrate;

d) a protein such as bovine serum albumin (BSA), gelatin, casein, immunoglobulin G (IgG);

e) a stabilizer such as glycerin, polyethylene glycol (PEG), propylene glycol (PG), polyvinyl alcohol (PVA);

f) a detergent such as an emulsifier, a surfactant, a wetting agent, and/or a defoaming agent. Tween® 20, Tween80®, NP-40 are examples of nonionic detergents and emulsifiers. Typical defoaming agents are polysiloxanes like Polydimethylsiloxane (PDMS) or esters made from fatty acids, simple or polyol alcohols and amines.

g) an enzyme such as protease;

h) a solvent such as a clearing agent (e.g. xylene, Histo-Clear® (National Diagnostics Inc., Cat. # HS-200), different types of organic solvents), propanol (propyl alcohol), ethanol (ethyl alcohol), methanol (methyl alcohol), aliphatic hydrocarbons, a food oil, vegitable oils, esters of fatty acids; and/or

i) another ingredient such as hydrogene peroxide.

The produced liquid product 123, 123′ may for example be a buffer solution, an alcohol solution or a diluted alcohol solution, a washing solution, or a solution of a combination of two or more of the above-mentioned ingredients.

In embodiments, the ingredient 113, 115, 117, 119 may be in liquid phase or in solid phase. For example, the ingredient 113, 115, 117, 119 may be a liquid, a concentrate, a powder, a paste, or a slurry.

FIG. 7 schematically illustrates an embodiment of a second ingredient source 114′ for a powder ingredient 115′. The shown ingredient source 114′ is of a so-called screw feeder type comprising an internal screw 121, such as an Archimedian screw. The ingredient source 114′ is arranged to be controlled by the production controller 134. The internal screw 121 is turned by a motor (not shown) whereby the ingredient 115′ is transported from the bottom part of the source 114′ to an upper part of the source 114′ where it is supplied to the supply conduit 130. The production controller 134 may control the second ingredient source 114′ to meter a predetermined amount of the ingredient 115′ for supply to the mixer station 120 via supply conduit 130.

It should be understood that the ingredient may be provided as a tablet or capsule, and that the ingredient source may be a tablet or capsule dispenser configured to dispense one or more tablets or capsules at a time. Providing the ingredient as a tablet or capsule may be especially advantageous when the ingredient comprises a freeze-dried enzyme.

Embodiments may further comprise a water tap or faucet 104, water conduit 105 and a water supply regulator 103 for regulating the supply of water to the first ingredient source 112 or to the mixer station 120. The water supply regulator 103 could be arranged in communication with the production controller 134, whereby the production controller 134 may be configured to control the operation of the water supply regulator 103.

Embodiments may further comprise a purifier 106 arranged to purify water transported in water conduit 105 and supplied to the purifier 106. In such embodiments the water supply regulator 103 is configured to control the supply of water to the purifier 106.

It should be understood that embodiments may comprise a bypass conduit 108 arranged to bypass the purifier 106, whereby water from the water tap 104 can be supplied directly to the production unit 110 via a bypass conduit 108. The supply flow regulator 103 controls whether water is to be supplied to the purifier 106 or is to bypass the purifier 106. This control may be accomplished in dependence of instructions from the production controller 134.

In some embodiments, a washing solution for washing components of the system is to be produced. In such embodiments, the production controller 134 may instruct the water tap 104 to supply water to the tap water quality control station 101 that determines the quality of the tap water. Information about the quality of the tap water is transmitted to the production controller 134 that determines if the quality of the water is good enough for the specific application, i.e. to produce washing solution. If the quality is good enough, the production controller 134 instructs the supply flow regulator 103 to supply the tap water directly to the production unit 110 via the bypass conduit 108.

In other cases, the production controller 134 may determine that the tap water supplied to the quality control station 101 is not of the required or desired quality, whereby the production controller 134 may instruct the supply flow regulator 103 to supply the tap water to the purifier 106 in order to purify the tap water. The purified tap water is then supplied to the production unit 110 and the quality may be checked by a first quality control station 107. If the purified tap water does not fulfill the quality requirements it can be transported through the production unit 110 to the waste container 166. A user may also be alerted, as will be described below, to check the water tap 104 and/or the purifier 106 and/or to replace the purifier 106 and/or one or more parts of the purifier 106.

The purifier 106 may be a filtration device, such as a Milli-Q® device from Millipore Corporation, a Cascada™ device from PALL Corporation, or another purification device. The purifier may also be a separator (not shown) configured to use reverse osmosis to purify the water. The purifier 106 could be arranged in communication with the production controller 134, whereby the production controller 134 may be configured to control the operation of the purifier 106.

In embodiments, the biological sample processing system 100 comprises at least one quality control station 101, 107, 152, 152′, 154, 164 arranged to check the quality of liquid supplied to the production unit 110, supplied within the production unit 110 and/or supplied from the production unit 110.

The at least one quality control station 101, 107, 152, 152′, 154, 164 may be arranged in communication with the production controller 134, whereby information and instructions can be transferred between the quality control station and the production controller.

It should be understood that the number of quality control stations may vary and may for example be dependent on the number of liquid inlets, e.g. water taps or other liquid sources, to the production unit; the number of mixer stations; or of the number of liquid storage stations comprised in the production unit 110. Further, it should be understood that quality control stations may also be arranged to control the quality of the ingredients supplied from the different ingredient sources comprised in the production unit 110.

A tap water quality control station 101 may be arranged in the water conduit 105 and may be configured to check the quality of the tap water from the water tap 104.

A first quality control station 107 may be arranged in communication with the water conduit 105 and may be configured to determine the quality of the water supplied to the production unit 110. In FIG. 1, the first quality control station 107 is shown to be arranged internally of the production unit 110, but it should be understood that the first quality control station 107 could be arranged externally of the production unit 100.

A second, third, fourth and/or an N^{th} quality control station 152, 154, 164 and/or 152′ may be arranged in the production unit 110 in order to determine the quality of liquid in the production unit 110.

The second quality control station 152, for example, may be arranged at a delivery conduit 132 and configured to determine the quality of liquid leaving the mixer station 120.

For example, the third quality control station 154 may be arranged at a storage inlet conduit 151 and configured to determine the quality of liquid entering a liquid storage station 156.

The fourth quality control station 164 may be arranged at a storage outlet conduit 161 and configured to determine the quality of the liquid leaving the liquid storage station 156.

The N^{th} quality control station 152′ may be arranged at an N^{th} delivery conduit 132′ and configured to determine the quality of liquid leaving the N^{th} mixer station 120′.

For example, the at least one quality control station 101, 107, 152, 152′, 154, 164 may be configured to determine the quality of the liquid by determining the pH of the liquid, by determining the color of the liquid and comparing the determined color with reference colors, or by determining the electrical conductivity of the liquid. If the quality is not within acceptable limits, the production controller 134 may continue or abort or pause the liquid production as desired. The production controller 134 may also be configured to alert or notify a user by sending quality information to the system manager SM 144. The alert or notification may be audible, visual or audiovisual. Quality information may be displayed on a display connected to or comprised in the system manager SM 144. The user may be alerted to check or replace parts of the system.

In an embodiment, at least one of the ingredient sources 112, 114, 116, 118 is arranged above the mixer station 120, 120′ such that an amount of at least one of the ingredients 113, 115, 117, 119 may be fed under gravity to the mixing chamber 122, 122′ from the ingredient source 112, 114, 116, 118.

In embodiments, the production unit 110 comprises a first supply flow regulator 146 arranged at the first supply conduit 128. The first supply regulator 146 may be controlled by the production controller 134 and arranged to supply an amount of the first ingredient 113 to the mixing chamber 122 in response to control instructions from the production controller 134. The production unit 110 may further comprise a second supply flow regulator 148 arranged at the second supply conduit 130. The second supply flow regulator 148 may be arranged to be controlled by the production controller 134 and arranged to supply an amount of the second ingredient 115 to the mixing chamber 122 in response to control instructions from the production controller 134. A delivery flow regulator 150 may be arranged at the delivery conduit 132 and arranged to be controlled by the production controller 134. The delivery flow regulator 150 may be arranged to transport an amount of the liquid product 123 from the mixing chamber 122 in response to control instructions from the production controller 134.

In embodiments, at least one of the flow regulators 146, 148, 150, 146′, 148′, 150′ comprised in the system comprises a pumping device, such as a self-priming pump or a metering pump. The metering pump may be a piston metering pump, diaphragm metering pump or a peristaltic metering pump. The pumping device may configured to pump a fluid at a constant flow velocity, i.e. to pump a constant volume per time unit, whereby it is possible to control the fluid volume pumped by controlling the time the pumping means is pumping the fluid.

In embodiments, at least one of the flow regulators 146, 148, 150, 146′, 148′, 150′ comprised in the system comprises a flow regulating valve, such as two-way valves, three-way valves or a four-way valve. The valve could be controlled by an electro-mechanical actuator such as an electric motor or solenoid, a pneumatic actuator controlled by air pressure, or a hydraulic actuator controlled by the pressure of a liquid such as oil or water.

In embodiments, the ingredient source 112, 114, 116, 118 could be connected to the respective supply flow conduit 128, 130, 128′, 130′ or to the respective supply flow regulator 146, 148, 146′, 148′ by means of a bayonet connector (not shown). The ingredient source 112, 114, 116, 118, may also be provided with a seal (not shown) that breaks when the ingredient source 112, 114, 116, 118 is connected to the respective supply flow conduit 128, 130, 128′, 130′ or to the respective supply flow regulator 146, 148, 146′, 148′.

In embodiments and especially in embodiments wherein at least one of the ingredients is a powder ingredient, the supply flow regulator 146, 148, 146′, 148′ for supplying the powder ingredient is a screw conveyor (not shown) comprising an internal screw, e.g. an Archimedian screw, and arranged between the outlet of the respective ingredient source 112, 114, 116, 118 and the inlet 124, 124′ of the respective mixing station 120, 120′. The screw conveyor is configured to supply a metered amount of the ingredient from the ingredient source 112, 114, 116, 118 to the respective mixing chamber 122, 122′ of the mixing station 120, 120′ upon rotation of the internal screw. The screw conveyor is further arranged to be controlled by the production controller 134 and configured to supply a controlled amount of the ingredient to the mixing station 120, 120′ in response to control instructions from the production controller 134.

In embodiments, at least one of the supply flow regulators is a shaking/vibrating device (not shown), such as a vibratory feeder, arranged at the ingredient source 112, 114, 116, 118 and configured to vibrate the ingredient source 112, 114, 116, 118 in response to control instructions from the production controller 134, whereby an amount of the ingredient 113, 115, 117, 119 is fed from the ingredient source 112, 114, 116, 118 to the mixing chamber 122, 122′.

In embodiments, at least one of the supply flow regulators is a source of pressurized air (not shown), such as a compressor (not shown), arranged at the ingredient source 112, 114, 116, 118 and configured to supply pressurized air to the ingredient source 112, 114, 116, 118 in response to control instructions from the production controller 134, whereby an amount of the ingredient 113, 115, 117, 119 is fed from the ingredient source 112, 114, 116, 118 to the mixing chamber 122, 122′.

In embodiments, the delivery flow regulator 150, 150′ is configured to transport an amount of the liquid product 123, 123′ to a quality control station 152, 152′, 154 arranged to control the quality of the liquid product 123, 123′. The delivery flow regulator 150, 150′ is further configured to transport quality controlled liquid product 123, 123′ to a production unit outlet 153 or to a liquid storage station 156 for storage.

In embodiments, the liquid storage station 156 comprises a storage chamber 157 having a storage inlet 158 and a storage outlet 159. The storage inlet 158 is connected, via storage inlet conduit 151, to a third quality control station 154 and the delivery flow regulator 150. The storage outlet 159 is connected, via storage outlet conduit 161, to a storage outlet flow regulator 163, a fourth quality control station 164 and to a production unit outlet 153. The liquid storage quality control station 164 may be arranged at the storage outlet conduit 161 and is configured to check the quality of the liquid product 123 that has been stored in the liquid storage station 156.

It should be understood that the number of liquid storage stations may vary and that a liquid storage station may be connected to a mixing station or to another liquid storage station for transportation of a liquid product between a mixing station and a liquid storage station, between two mixing stations or between two liquid storage stations. The liquid storage station may also be configured as a mixer station.

In embodiments, an antimicrobial device 155 may be arranged in the liquid storage station 156 and configured to prevent bacterial growth in the liquid storage station 156. The antimicrobial device 155 may comprise a heat source or a UV source, whereby bacterial growth is prevented by heat or UV radiation.

The production unit outlet 153, such as a manifold, is connected to a liquid product tap 165 by means of which an amount of the liquid product 123 can be dispensed from the production unit 110. The liquid product tap 165 provides a user, such as a laboratory technician, to withdraw a desired volume of a liquid product from the production unit 110 in a controlled manner.

It should be understood that the liquid product tap 165 illustrated in FIG. 1 may comprise several taps or nozzles, e.g. one tap or nozzle for each produced liquid product.

In embodiments, the biological sample processing system comprises one or more fluidics carts 167 having one or more fluid containers 168, sometimes also called liquid product containers. The one or more of the fluid containers 168 of the one or more fluidics cart 167 may be connected to the production unit outlet 153, whereby liquid product 123, 123′ can be supplied from the production unit 110 to the one or more of the fluid containers 168. The fluidics cart 167 may be arranged in communication with the sample processing apparatus controller SC 142 that is configured to control the supply of fluid from the fluid container 168 to the sample processing apparatus 102.

In embodiments, weighing means (not shown) could be arranged in the fluid cart 167 to weigh the fluid containers 168 and thereby determine the amount of fluid comprised in each of the container 168. The weighing means may be similar to the weighing means in the mixer station as described below. The weighing means may be a weighing scale or a balance. When the weighing means determines that the amount of fluid is below a minimum volume it could be configured to inform the sample processing apparatus controller SC 142 which may send instructions to the production controller 134 to produce more of the fluid.

In embodiments, the fluidic containers 168 may be provided with one or more level sensing mechanism, such as level sensors (not shown), for sensing the level of a fluid in the container 168. The level mechanism may be similar to the level mechanism in the mixer station as described below. The level sensor could be a conductive electrode-based level sensor or a microwave/radar level sensor. The level sensor may be arranged to sense a minimum level of fluid in the container 168. When the fluid is below the minimum level, the level sensor may be configured to inform the sample processing apparatus controller SC 142 which may send instructions to the production controller 134 to produce more of the fluid.

In embodiments, instructions may be sent from the sample processing apparatus controller SC 142 directly to the production unit 134, but instructions may also be sent from the sample processing apparatus controller SC 142 indirectly to the production unit 134 via a system manager server SMS 140.

Depending on the relevance of the fluid for the sample processing the production of the fluid may be assigned a priority. For example, if the fluid has high relevance for the sample processing, the production of the fluid may be assigned a high priority, whereby the production of that fluid will be higher prioritized as compared to the production of other fluids having a lower priority. In embodiments, the scheduler is configured to accomplish the prioritization between different liquids to be produced. The prioritization may change during the operation of the sample processing apparatus since new high priority samples, e.g. stat samples, may be loaded in the apparatus, and the processing of which samples requires a certain processing fluid that may have to be produced before the processing of the samples can be accomplished.

In embodiments, a weighing means 160, 160′ may be arranged at the mixer station 120, 120′ and arranged in communication with the production controller 134. The weighing means 160, 160′ may be configured to weigh the mixing chamber 122, 122′ to determine the amount of ingredient 113, 115, 117, 119 comprised in the mixing chamber 122, 122′ and configured to communicate the amount of ingredient to the production controller 134, whereby the production controller 134 can determine if more ingredient 113, 115, 117, 119 is to be supplied or if a sufficient amount of ingredient has been supplied and whereby the production unit controller 134 can control the production unit 110 accordingly. The weighing means 160, 160′ may be a weighing scale or a balance.

In embodiments, at least one level sensing mechanism 162, 162′ may be arranged at the mixer station 120, 120′ and arranged in communication with the production controller 134. The level sensing mechanism 162, 162 may be configured to sense the filling level in the mixing chamber 122, 122′ and configured to communicate the filling level to the production controller 134.

It should be understood that more than one level sensing mechanism 162, 162′ may be arranged at one and the same mixer station 120, 120′, whereby the different level sensing mechanism 162, 162′ may be arranged to determine different filling levels within the same mixer station 120, 120′.

In embodiments, the level sensing mechanism 162 is a level sensor 162 such as a conductive electrode-based level sensor or a microwave/radar level sensor.

In embodiments, the ingredient source 112, 114, 116, 118 is a disposable and/or collapsible container 112, 114, 116, 118. The container 112, 114, 116, 118 may be provided with quick release coupling means (not shown) to enable them to be easily attached to and removed from the production unit 110. The quick release coupling means may be a bayonet mount/bayonet connector, or another fastening means providing quick and easy mounting and demounting. The fastening means may rely on mated surfaces; a male side with one or more pins or slots, and a female receptor with matching slots and a spring that maintains a clamping force.

In embodiments, the ingredient source 112, 114, 116, 118 is configured as a flexible bag comprising the ingredient 113, 115, 117, 119. The flexible bag may be comprised in a box in a so-called bag-in-a-box configuration. The flexible bag may further be configured to prevent air from entering into the bag.

In embodiments, at least one of the ingredient sources 112, 114, 116, 118 has a rigid construction and is open to the surrounding atmosphere.

In embodiments, the liquid product 123, 123′ may be produced as a batch or may be produced continuously. The liquid product 123, 123′ may also be produced on demand.

In embodiments, the mixing chamber 122, 122′ is a disposable and/or collapsible container provided with quick release coupling means, such as a bayonet connector (not shown), to enable it to be easily attached to and removed from the production unit 110.

In embodiments, the mixing chamber 122, 122′ is a stationary container arranged in the production unit 110. In such embodiments, the mixing chamber 122, 122′ may be rinsed between two liquid production processes. For example, the mixing chamber may be rinsed with a washing solution supplied from an ingredient source via one or more conduits to the mixing chamber. In embodiments, a flow of washing solution through the mixing chamber is provided for a defined time period. After which time period, the mixing chamber may be considered to be clean and a flow of e.g. purified water may be provided through the mixing chamber to remove possible residuals of the washing solution.

In some embodiments, the ingredient sources, and/or mixing stations, and/or liquid product containers are each provided with an identification label (not shown), preferably a unique identification label. By means of the identification label, information relating to the ingredient source, the mixing station, and the liquid product container, respectively, may be retrieved from a data storage or may be read electronically from the label. The identification label may be an optical identification element. In some embodiments, one type of optical identification element may be optical character recognition or a two-dimensional symbology, such as the so-called “Infoglyph™” type or other identification element such as RF tags. In some embodiments, one or more optical and/or electronic sensors may be used to retrieve e.g. ingredient or liquid product information.

The optical identification element may be an adhesive label carrying encoded information about the content of the ingredient source, mixing station or the liquid product container, such as ingredient type, liquid product, date of manufacture, expiry date, and/or a unique identification number that identifies the ingredient source, mixing station or the liquid product container such as in a central networked database. The encoded information may be in the form of a data matrix code, an Infoglyph™ code or any other kind of two-dimensional (2-D) code, and could in principle also be a simple one-dimensional (1-D) code, i.e. a bar code. The aforementioned encoded information may correspond, for example, to unique identification which may be utilized, for instance, to retrieve certain data from a central database server. Additionally, the optical identification element or label may also be provided with human readable text to aid the operator handling the ingredient source, mixing station, and/or liquid product containers e.g. during loading of ingredient source into the production unit or during loading liquid product containers to the fluidics chart. Additional types of identification may also be employed for ingredient sources and/or mixing stations and/or liquid product containers such as utilizing Radio Frequency (RF) tag or RF data carrier technology (examples, of which, are described in U.S. Pat. Nos. 6,941,202, 6,922,146, 6,883,710). Additional alternatives include but are not limited to identifying samples as described in U.S. patent application. Ser. No. 11/168,987, the contents of which are hereby incorporate by reference.

In embodiments, the production controller 134 may be configured to control the production unit 110 to produce the liquid product 123, 123′ in dependence on the consumption of the liquid product 123, 123′ by a sample processing apparatus 102 connected to the production unit 110.

In embodiments, the production unit 110 is comprised in the sample processing apparatus 102.

In embodiments, the production unit 110 is connected to more than one sample processing apparatuses 102.

In embodiments, the biological sample processing system may comprise additional conduits and additional flow regulators 169, 170, 171, 172 for supplying an ingredient from one of the ingredient sources to any of the supply conduits 128, 130, 128′, 130′ and to any of the mixer stations 120, 120′. For example, by this arrangement, water from the water tap may be supplied to all the supply conduits of the system, to all the mixer stations and storage stations and further through the delivery conduits to the waste container. Another example is that a washing solution comprised in one of the ingredient sources may be supplied through any of the supply conduits, any of the mixer stations, any of the storage stations, and through the delivery conduits to the waste container, whereby the desired conduits and stations may be washed.

In embodiments, the conduits, the mixing station, and storage stations may be emptied by transporting, e.g. pumping, their content to the waste container 166, e.g. via the production unit outlet 153.

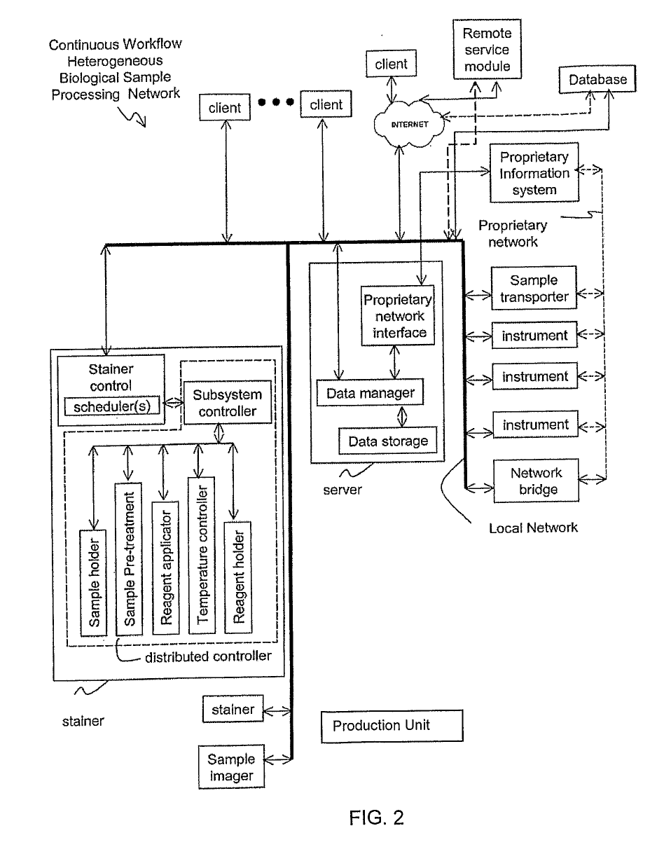

FIG. 2 is an overview of one embodiment of a continuous workflow heterogeneous biological sample processing network comprising a liquid production unit as described herein. In one embodiment, a heterogeneous network is not required. For example, one embodiment of the invention is a continuous workflow biological sample processing apparatus. Not all elements of the embodiment are required to achieve at least one of the advantages described herein. Examples of each of these elements and examples of how they interact and operate are provided herein. For example, the embodiment may be a continuous workflow slide staining system, however it is not limited to a staining apparatus. The stainer may have a plurality of sample holders and/or reagent holders. The stainer may also comprise a distributed controller which may control at least one element chosen from at least one sample holder, at least one sample pre-treatment element, at least one reagent applicator, at least one temperature controller, and at least one reagent holder. A scheduler may schedule tasks, such as scheduling production of liquid products in dependence of the priority of the respective liquid product to be produced. In one embodiment, the scheduler(s) may be non-deterministic and adaptive, i.e. does whatever is next and whatever is next may change for a variety of conditions. In one embodiment, the stainer communicates with a network, such as a heterogeneous network. The network, in one embodiment, comprises a computer that will provide data storage and retrieval, data manager, and data communications. As shown in FIG. 2, the network may include clients for monitoring & receiving, view, inputting, programming, analyzing, and editing, data to the system and it may also include other instrumentation for further handling, sorting, processing and analysis of samples. The network may also include a laboratory information system LIS interface that provides ability to communicate with proprietary networks. Alternative embodiments and more specific embodiments of such a heterogeneous biological sample processing network are described as follows.

FIG. 3 illustrates an embodiment of a continuous workflow heterogeneous biological sample processing network 5300. One additional embodiment of the invention is a continuous workflow biological sample apparatus. A server PC 5316, which includes System Manager Server (SMS) software 5314, is connected to a network 5318. Network 5318 may be an Ethernet 10/100 base T network, a wireless network, such as 802.11b, or any desired network. For purposes of this application, server 5316 may be implemented on a specific computer or on a plurality of computer such as a cluster. System Manager Server (SMS) 5314 communicates via network 5318 with Stainer Control Software (SCS) 5352 that controls and monitors various pre-treatment, processing and staining functions that are implemented on a first stainer 5320.

Other stainers such as a second stainer 5322, or any desired number of stainers up to an Nth stainer 5324, may be connected to network 5318 so that System Manager Server (SMS) software 5314 may communicate with any of the stainers 5320, 5322, and 5324 that are connected to network 5318. A network 5318 that connects a System Manager Server (SMS) 5314 to only stainers may be thought of as a homogeneous network, i.e., a network that connects a server to processing apparatuses of the same type in accordance with one embodiment of the invention. An additional embodiment may include more than one network i.e., one dedicated LAN and perhaps a LAN of a remote laboratory which may be further connected via a router and/or a bridge device. This may also provide connectivity to the LIS Agent 5312. Additionally, client capability interactivity, such as for personnel deployment, may be realized via the remote LAN of a laboratory, for example, which may also access the SMS.

In the embodiment of FIG. 3, a heterogeneous biological sample processing network 5300 may be implemented, i.e. a network that connects a System Manager Server (SMS) 5314 to different types of instruments. For example, network 5318 may connect to sample processing equipment such as slide image 5326 which may be a slide imager such as the ACIS family of imagers from CLARiENT, Inc, San Juan Capistrano, Calif. Other instrument types related to sample processing may include one or more liquid production units 110, an automated microtome 5340, tissue processor 5342, special stainer 5344, and in-situ hybridization stainer 5346.

Still other types of instruments, such as flow cytometry analyzer 5348 and flow cytometer sorter 5350 may also be adapted to connect to server 5316 and to System Manager Server (SMS) 5314. Each of the different instruments 5326, 5340, 5342, 5344, 5346, 5348, 5350, may be equipped with desired network hardware and software that enables System Manager Server (SMS) to request data from or send data to any instrument that is connected to network 5318.

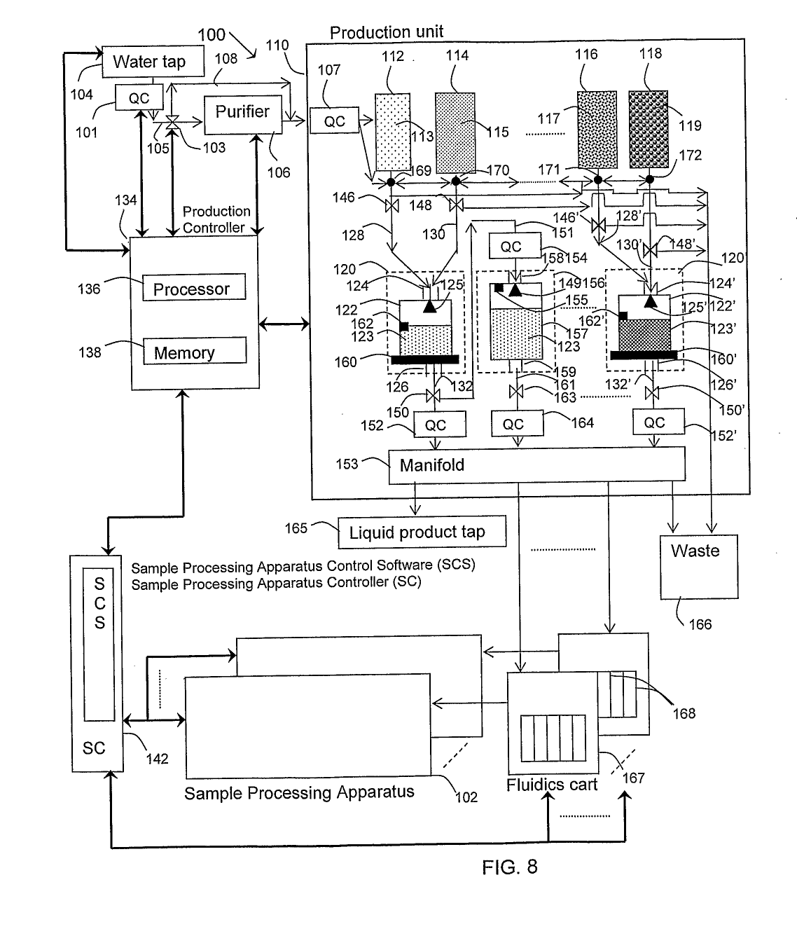

FIG. 8 schematically shows an alternative embodiment of a biological sample processing system. As previously described, in embodiments, the biological sample processing system 100 comprises a production unit 110 connected to the biological sample processing apparatus 102. The biological sample processing system 100 comprises further a production controller 134 arranged in communication with the production unit 110 and configured to control the production unit 110. In embodiments, the production controller 134 may be comprised in the production unit 110.

The production controller 134 comprises a processor 136, and a memory device 138 in communication with the processor 136. In embodiments, the sample processing system 100 may further comprise a sample processing apparatus controller SC 142 arranged in communication with the sample processing apparatus 102 and the production controller 134. The sample processing apparatus controller SC 142 comprises sample processing apparatus control software SCS and is configured to control and monitor various processes, functions, or components, etc. which are implemented on or relate to the operation or control of the sample processing apparatus 102. The sample processing apparatus controller SC 142 may be configured to transfer data to and from the production controller 134.

Systems and methods for on-site liquid production, and especially to biological sample processing systems and methods for on-site liquid production, have been disclosed and described according to some explanatory embodiments. Those skilled in the art can now appreciate, from the foregoing description, that the broad techniques of the embodiments of the present invention can be implemented in a variety of forms. Therefore, while the embodiments of this invention have been described in connection with particular examples thereof, the true scope of the embodiments of the present invention should not be so limited since many variations and equivalents of the method and the apparatus may be carried out without departing from the scope of the invention.

Examples of the invention include, but are not limited to, the description in the following paragraphs:

Paragraph 1. A biological sample processing system (100), comprising:

a biological sample processing apparatus (102) for processing of biological samples arranged on microscope slides;

a production unit (110) connected to the biological sample processing apparatus (102), the production unit (110) comprising:
a first ingredient source (112) of a first ingredient (113);
a second ingredient source (114) of a second ingredient (115);
a mixer station (120) comprising a mixing chamber (122) having a mixer inlet (124) and a mixer outlet (126), the mixer station (120) being configured to mix ingredients in the mixing chamber (122) to produce a liquid product (123);
a first supply conduit (128) for supply of an amount of the first ingredient (113) from the first ingredient source (112) to the mixing chamber (122) via the mixer inlet (124);
a second supply conduit (130) for supply of an amount of the second ingredient (115) from the second ingredient source (114) to the mixing chamber (122) via the mixer inlet (124); and
a delivery conduit (132) for transportation of an amount of the liquid product (123) from the mixing chamber (122) via the mixer outlet (126); and

a production controller (134) arranged in communication with the production unit (110), the production controller (134) comprising:
a processor (136); and
a memory device (138) in communication with the processor (136), the memory device (138) comprising a computer program which the processor (136) is configured to access to control the operation of the production unit (110).

Paragraph 2. The biological sample processing system of paragraph 1, further comprising a sample processing apparatus controller SC (142) arranged in communication with the sample processing apparatus (102), and being configured to control and monitor various processes, functions, or components, that are implemented on or relates to the operation of the sample processing apparatus (102).

Paragraph 3. The biological sample processing system of paragraph 2, wherein the sample processing apparatus controller SC (142) is arranged to either directly or indirectly schedule the production of liquids in the production unit (110).

Paragraph 4. The biological sample processing system of paragraph 2, further comprising a system manager server SMS (140) arranged in communication with the production controller (134) and the sample processing apparatus controller SC (142), and configured to control the production controller (134).

Paragraph 5. The biological sample processing system of paragraph 4, wherein the sample processing apparatus controller SC (142) is configured to transfer data to and from the system manager server SMS (140).

Paragraph 6. The biological sample processing system of paragraph 4 or 5, further comprising a system manager SM (144) arranged in communication with the system manager server SMS (140) and configured to provide a user to connect to, and to transfer data to and from the system manager server SMS (140).

Paragraph 7. The biological sample processing system of paragraph 6, wherein the system manager SM (144) is comprised in a client PC (5302).

Paragraph 8. The biological sample processing system of any of paragraph 4-7, wherein the production controller (134) comprises the system manager server SMS (140).

Paragraph 9. The biological sample processing system of paragraph 1, wherein the first ingredient source (112) is a water container and the first ingredient (113) is water.

Paragraph 10. The biological sample processing system of paragraph 1, wherein the mixer inlet (124) is provided with a spray nozzle (125) configured to supply an amount of the ingredient (113, 115) to the mixing chamber (122) in a spray pattern (127).

Paragraph 11. The biological sample processing system of paragraph 1, wherein the mixer station (120) comprises a mixing means (129a, 129b) rotatably arranged within the mixing chamber (122) and configured to mix the ingredients in the mixing chamber (122) upon rotation.

Paragraph 12. The biological sample processing system of paragraph 1, wherein the mixer station (120) comprises a vortex mixer (129c) configured to mix the ingredients in the mixing chamber (122) upon rotation.

Paragraph 13. The biological sample processing system of paragraph 1, wherein the second ingredient source (114) comprises an internal screw (121) controlled by the production controller (134) and configured to meter a predetermined amount of the second ingredient (115) for supply to the mixer station (120).

Paragraph 14. The biological sample processing system of paragraph 1, further comprising a water tap (104), water conduit (105) and a water supply regulator (103) for supplying an amount of water to the first ingredient source (112) or to the mixer station (120).

Paragraph 15. The biological sample processing system of paragraph 14, further comprising a purifier (106) arranged to purify the water transported in water conduit (105).

Paragraph 16. The biological sample processing system of paragraph 15, wherein the purifier (107) is a filtration device or a separator configured to use reverse osmosis to purify the water.

Paragraph 17. The biological sample processing system of paragraph 1 further comprising at least one quality control station (101, 107, 152, 152′, 154, 164) arranged to control the quality of liquid supplied to, supplied within or supplied from the production unit (110).

Paragraph 18. The biological sample processing system of paragraph 17, wherein the at least one quality control station (101, 107, 152, 152′, 154, 164) is configured to determine the pH of the liquid.

Paragraph 19. The biological sample processing system of paragraph 17, wherein the at least one quality control station (101, 107, 152, 152′, 154, 164) is configured to determine the quality of the liquid by determining the color of the liquid and by comparing the determined color with reference colors.

Paragraph 20. The biological sample processing system of paragraph 17, wherein the at least one quality control station (101, 107, 152, 152′, 154, 164) is configured to determine the electrical conductivity of the liquid.

Paragraph 21. The biological sample processing system of paragraph 1, wherein the second ingredient (115) is a liquid, a concentrate, a powder, a paste or a slurry.

Paragraph 22. The biological sample processing system of paragraph 1, wherein at least one of the first ingredient source (112) and second ingredient source (114) is arranged above the mixer station (120), whereby an amount of the first ingredient (113) and/or an amount of the second ingredient (115) is fed under gravity to the mixing chamber (122) from the first ingredient source (112) and/or the second ingredient source (114), respectively.

Paragraph 23. The biological sample processing system of paragraph 1, further comprising:
a first supply flow regulator (146) arranged at the first supply conduit (128), arranged to be controlled by the production controller (134) and arranged to supply an amount of the first ingredient (113) to the mixing chamber (122) in response to control instructions from the production controller (134);
a second supply flow regulator (148) arranged at the second supply conduit (130), arranged to be controlled by the production controller (134) and arranged to supply an amount of the second ingredient (115) to the mixing chamber (122) in response to control instructions from the production controller (134); and
a delivery flow regulator (150) arranged at the delivery conduit (132), arranged to be controlled by the production controller (134) and arranged to transport an amount of the liquid product (123) from the mixing chamber (122) in response to control instructions from the production controller (134).

Paragraph 24. The biological sample processing system of paragraph 23, wherein at least one of the first supply flow regulator (146), the second supply flow regulator (148) and the delivery flow regulator (150) comprises a pumping device and a flow regulating valve.

Paragraph 25. The biological sample processing system of paragraph 23, wherein the supply flow regulator (146, 148) is opened when the ingredient source (112, 114) is put in its place.

Paragraph 26. The biological sample processing system of paragraph 23, wherein the first supply flow regulator (146) and/or the second supply flow regulator (148) is a screw conveyor arranged between the outlet of the respective first ingredient source (112) and/or second ingredient source (114) and the inlet (124) of the mixing chamber (122), and configured to supply an amount of the respective first ingredient (113) and/or second ingredient (115) to the mixing chamber (122).

Paragraph 27. The biological sample processing system of paragraph 23, wherein the second supply flow regulator (148) is a vibrating device, such as a vibratory feeder, configured to vibrate the second ingredient source (114) in response to control instructions from the production controller (134), whereby an amount of the second ingredient (115) in the second ingredient source (114) is fed from the second ingredient source (114) to the mixing chamber (122).

Paragraph 28. The biological sample processing system of paragraph 23, wherein the second supply flow regulator (148) is a source of pressurized air, such as a compressor, configured to supply pressurized air to the second ingredient source (114) in response to control instructions from the production controller (134), whereby an amount of the second ingredient (115) in the second ingredient source (114) is fed from the second ingredient source (114) to the mixing chamber (122).

Paragraph 29. The biological sample processing system of paragraph 23, wherein the second supply flow regulator (148) is an internal screw mixer arranged in the second ingredient source (114) and configured to provide a metered supply of the second ingredient (115) from the second ingredient source (114) to the mixing chamber (122) in response to control instructions from the production controller (134).

Paragraph 30. The biological sample processing system of paragraph 23, wherein the delivery flow regulator (150) is configured to transport an amount of the liquid product (123) to a second or third quality control station (152, 154) arranged to control the quality of the liquid product (123), and configured to transport quality controlled liquid product (123) to a production unit outlet (153) or to a liquid storage station (156) for storage.

Paragraph 31. The biological sample processing system of paragraph 30, wherein the liquid storage station (156) comprises a storage chamber (157) having:
a storage inlet (158) connected, via storage inlet conduit (151), to the third quality control station (154) and the delivery flow regulator (150); and
a storage outlet (159) connected, via storage outlet conduit (161), to a fourth quality control station (164) and to a production unit outlet (153).
Paragraph 32. The biological sample processing system of paragraph 31, further comprising an antimicrobial device (155) arranged in the liquid storage station (156) and configured to prevent bacterial growth in the liquid storage station (156).

Paragraph 33. The biological sample processing system of paragraph 32, wherein the antimicrobial device (155) comprises a heat source or a UV source, whereby bacterial growth is prevented by heat or UV radiation.

Paragraph 34. The biological sample processing system of any of paragraph 30-33, further comprising a liquid storage quality control station (164) for controlling the quality of the liquid product (123) that has been stored in the liquid storage station (156).

Paragraph 35. The biological sample processing system of paragraph 34, wherein the liquid storage quality control station (164) is arranged at the storage outlet conduit (161).

Paragraph 36. The biological sample processing system of any of paragraph 30-35, wherein the production unit outlet (153) is connected to a liquid product tap (165) by means of which an amount of the liquid product (123) can be withdrawn from the production unit (110).

Paragraph 37. The biological sample processing system of any of paragraph 30-36, further comprising one or more fluidics carts (167) comprising one or more fluid container (168), the fluidics cart (167) is connected to the production unit outlet (153), whereby liquid product (123) can be supplied from the production unit (110) to one or more of the fluid containers (168) on one or more of the fluidics carts (167).

Paragraph 38. The biological sample processing system of paragraph 37, wherein the fluidics cart (167) is arranged in communication with the sample processing apparatus controller SC (142) that is configured to control the supply of fluid from the fluid container (168) on the fluidics cart (167) to the sample processing apparatus (102)

Paragraph 39. The biological sample processing system of paragraph 1, further comprising a weighing means (160) arranged in the mixer station (120) and arranged in communication with the production controller (134), the weighing means (160) is configured to weigh the mixing chamber (122) to determine the amount of ingredient comprised in the mixing chamber (122) and configured to communicate the amount of ingredient to the production controller (134), whereby the production controller (134) can determine if more ingredient is to be supplied or if sufficient ingredient has been supplied and whereby the production unit controller (134) can control the production unit (110) accordingly.

Paragraph 40. The biological sample processing system of paragraph 39, wherein the weighing means (160) is a weighing scale or a balance.

Paragraph 41. The biological sample processing system of paragraph 1, further comprising at least one level sensing mechanism (162) arranged in the mixer station (120) and arranged in communication with the production controller (134), the level sensing mechanism (162) being configured to sense the filling level in the mixing chamber (122) and configured to communicate the filling level to the production controller (134).

Paragraph 42. The biological sample processing system of paragraph 41, wherein the level sensing mechanism (162) is a level sensor (162) such as a conductive electrode-based level sensor or a microwave/radar level sensor.

Paragraph 43. The biological sample processing system of paragraph 1, wherein the first ingredient source (112) and second ingredient source (114) are disposable and/or collapsible containers (112, 114).

Paragraph 44. The biological sample processing system of paragraph 43, wherein the containers (112, 114) are provided with quick release coupling means to enable them to be removed from the production unit (110).

Paragraph 45. The biological sample processing system of paragraph 1, wherein at least one of the first and second ingredient sources (112, 114) is configured as a flexible bag comprising the first or second ingredient (113, 115).

Paragraph 46. The biological sample processing system of paragraph 45, wherein the flexible bag is comprised in a box in a so-called bag-in-a-box configuration.

Paragraph 47. The biological sample processing system of paragraph 45, wherein the flexible bag is configured to prevent air from entering into the bag.

Paragraph 48. The biological sample processing system of paragraph 1, wherein at least one of the first and second ingredient sources (112, 114) has a rigid construction and is open to the surrounding atmosphere.

Paragraph 49. The biological sample processing system of any of the preceding paragraph, wherein the liquid product (123) is produced as a batch or is produced continuously.

Paragraph 50. The biological sample processing system of any of the preceding paragraph, wherein the liquid product (123) is produced on demand.

Paragraph 51. The biological sample processing system of any of the preceding paragraph, wherein the first ingredient (113) is purified water, the second ingredient (115) is a buffer concentrate and the liquid product (123) is a buffer solution.

Paragraph 52. The biological sample processing system of paragraph 1, wherein the mixing chamber (122) is a disposable and/or collapsible container (122) provided with quick release coupling means to enable it to be removed from the production unit (110).

Paragraph 53. The biological sample processing system of paragraph 1, wherein the mixing chamber (122) is a stationary container arranged in the production unit (110).

Paragraph 54. The biological sample processing system of paragraph 1, wherein the production controller (134) is configured to control the production unit (110) to produce the liquid product (123) in dependence on the consumption of the liquid product (123) by the sample processing apparatus (102).

Paragraph 55. The biological sample processing system of paragraph 1, wherein the production unit (110) is comprised in the sample processing apparatus (102).

Paragraph 56. The biological sample processing system of paragraph 1, wherein the production unit (110) is connected to more than one sample processing apparatus (102).

Paragraph 57. The biological sample processing system of paragraph 1, further comprising an (L−1)^{th} source (116) comprising an (L−1)^{th} ingredient (117), the (L−1)^{th} source (116) being configured to supply an amount of the (L−1)^{th} ingredient (117) to the first mixer station (120) or to an N^{th} mixer station (120′) comprised in the production unit (110).

Paragraph 58. The biological sample processing system of paragraph 1, further comprising an L^{th} source (118) comprising an L^{th} ingredient (119), the L^{th} source (118) being configured to supply an amount of the L^{th} ingredient (119) to the first mixer station (120) or to an N^{th} mixer station (120′) comprised in the production unit (110).

Paragraph 59. The biological sample processing system of paragraph 57 or 58, wherein the N^{th} mixer station (120′) comprises a mixing chamber (122′) having a mixer inlet (124′) and a mixer outlet (126′), the mixer station (120′) being configured to mix ingredients in the mixing chamber (122′) to produce a liquid product (123′).

Paragraph 60. The biological sample processing system of paragraph 1, further comprising conduits and flow regulators (169, 170, 171, 172) configured to supply an amount of the first ingredient (113) from the first ingredient source (112) or an amount of water from the water tap (104) to the mixing chamber (122) of the mixing station (120) via the second supply conduit (130).

Paragraph 61. The biological sample processing system of paragraph 60, wherein an amount of the first ingredient (113) can be supplied from the first ingredient source (112) or an amount of water from the water tap (104) to an N^{th} mixing chamber (122′) of an N^{th} mixing station (120′) via an (L−1)^{th} conduit (128′).

Paragraph 62. A method for liquid production in a biological sample processing system (100), comprising the steps of:

providing a biological sample processing apparatus (102) for processing of biological samples arranged on microscope slides;

providing a production unit (110) connected to the biological sample processing apparatus (102), the production unit (110) comprising:
a first ingredient source (112) of a first ingredient (113);
a second ingredient source (114) of a second ingredient (115);
a mixer station (120) comprising a mixing chamber (122) having a mixer inlet (124) and a mixer outlet (126), the mixer station (120) being configured to mix ingredients in the mixing chamber (122) to produce a liquid product (123);

providing a production controller (134) arranged in communication with the production unit (110), the production controller (134) comprising:
a processor (136); and
a memory device (138) in communication with the processor (136), the memory device (138) comprising a computer program which the processor (136) is configured to access to control the operation of the production unit (110);

supplying an amount of the first ingredient (113) from the first ingredient source (112) to the mixing chamber (122) in dependence of control instructions from the production controller (134);

supplying an amount of the second ingredient (115) from the second ingredient source (114) to the mixing chamber (122) in dependence of control instructions from the production controller (134);

mixing in the mixer chamber (122) the amount of the first ingredient (113) and the amount second ingredient (115) in dependence of control instructions from the production controller (134) to produce the liquid product (123), and

transporting an amount of the liquid product (123) from the mixing chamber (122) via the mixer outlet (126).

Paragraph 63. The method of paragraph 62, further comprising the steps of:

providing a sample processing apparatus controller SC (142) in communication with the sample processing apparatus (102), and

controlling and monitoring various processes, functions, or components, that are implemented on or relates to the operation of the sample processing apparatus (102).

Paragraph 64. The method of paragraph 63, further comprising the step of either directly or indirectly scheduling the production of liquids in the production unit (110) by means of the sample processing apparatus controller SC (142).

Paragraph 65. The method of paragraph 63, further comprising the steps of:

providing a system manager server SMS (140) arranged in communication with the production controller (134) and the sample processing apparatus controller SC (142); and

controlling the production controller (134) by means of the system manager server SMS (140).

Paragraph 66. The method of paragraph 65, further comprising the step of transferring data to and from the system manager server SMS (140) by means of the sample processing apparatus controller SC (142).

Paragraph 67. The method of paragraph 65 or 66, further comprising the steps of:

providing a system manager SM (144) arranged in communication with the system manager server SMS (140);

providing a user to connect to, and to transfer data to and from the system manager server SMS (140) by means of the a system manager SM (144).

Paragraph 68. The method of paragraph 62, wherein the steps of supplying an amount of the ingredients (113, 115) to the mixing chamber (122) comprises the step of supplying an amount of the ingredient (113, 115) to the mixing chamber (122) in a spray pattern (127).

Paragraph 69. The method of paragraph 62, further comprising the step of providing a purifier (106) arranged to purify the water transported in water conduit (105).

Paragraph 70. The method of paragraph 62, further comprising the step of providing at least one quality control station (101, 107, 152, 152′, 154, 164) arranged to control the quality of liquid supplied to, supplied within or supplied from the production unit (110).

Paragraph 71. The method of paragraph 62, further comprising the steps of:

providing a liquid storage station (156); and

transporting an amount of the liquid product (123) from the mixing chamber (122) to the liquid storage station (156) for storage.

Paragraph 72. The method of paragraph 62, further comprising the steps of:

providing a production unit outlet (153) connected to a liquid product tap (165);

transporting an amount of the liquid product (123) from the mixing chamber (122) to the production unit outlet (153); and

withdrawing an amount of the liquid product (123) from the production unit (110) by means of the liquid production tap (165).

Paragraph 73. The method of paragraph 62, further comprising the steps of:

providing a production unit outlet (153) connected to one or more fluidics carts (167) comprising one or more fluid container (168), and

supplying an amount of the liquid product (123) from the production unit (110) to one or more of the fluid containers (168).

Paragraph 74. The method of paragraph 62, further comprising the step of producing the liquid product (123) as a batch or continuously.

Paragraph 75. The method of paragraph 62, further comprising the step of producing the liquid product (123) on demand.

Paragraph 76. The method of paragraph 62, further comprising the step of controlling the production unit (110) to produce the liquid product (123) in dependence on the consumption of the liquid product (123) by the sample processing apparatus (102).

Paragraph 77. A computer-readable medium that stores instructions, which when executed by a processor control steps in a method according to any of paragraph 62-76.

List of reference numerals

| | |
|---|---|
| 100 | biological sample processing system |
| 101 | tap water quality control station |
| 102 | sample processing apparatus(-es) |
| 103 | water supply regulator |
| 104 | water tap/faucet |
| 105 | water conduit |
| 106 | purifier |
| 107 | 1^{st} quality control station |
| 108 | bypass conduit |
| 110 | production unit |
| 112 | 1^{st} ingredient source |
| 113 | 1^{st} ingredient |
| 114, 114′ | 2^{nd} ingredient source |
| 115, 115′ | 2^{nd} ingredient |
| 116 | (L − 1)^{th} ingredient source |
| 117 | (L − 1)^{th} ingredient |
| 118 | L^{th} ingredient source |
| 119 | L^{th} ingredient |
| 120, 120′ | 1^{st}, N^{th} mixer station |
| 121 | internal screw |
| 122, 122′ | 1^{st}, N^{th} mixing chamber |
| 123, 123′ | liquid product, N^{th} liquid product |
| 124, 124′ | 1^{st}, N^{th} mixer inlet |
| 125, 125′ | 1^{st}, Nth nozzle |
| 126, 126′ | mixer outlet, N^{th} mixer outlet |
| 127, 127′ | spray pattern |
| 128, 128′ | 1^{st}, (L − 1)^{th} supply conduit |
| 129, 129′ | 1^{st}, N^{th} mixing means |
| 130, 130′ | 2^{nd}, L^{th} supply conduit |
| 134 | production controller |
| 132, 132′ | 1^{st}, N^{th} delivery conduit |
| 136 | processor |
| 138 | memory |
| 140 | system manager server SMS |
| 142 | sample processing apparatus |
| | controller SC |
| 144 | system manager SM |
| 146, 146′ | 1^{st}, (L − 1)^{th} supply flow regulator |
| 148, 148′ | 2^{nd}, L^{th} supply flow regulator |
| 149 | storage nozzle |
| 150, 150′ | 1^{st}, N^{th} delivery flow regulator |
| 151 | storage inlet conduit |
| 152, 152′ | 2^{nd}, N^{th} quality control station |
| 153 | production unit outlet, manifold |
| 154 | 3^{rd} quality control station |
| 155 | antimicrobial device |
| 156 | liquid storage station |
| 157 | storage chamber |
| 158 | storage inlet |
| 159 | storage outlet |
| 160, 160′ | weighing means |
| 161 | storage outlet conduit |
| 162, 162′ | level sensing mechanism |
| 163 | storage outlet flow regulator |
| 164 | 4^{th} quality control station |
| 165 | liquid product tap |
| 166 | waste container |
| 167 | fluidics cart |
| 168 | fluid container |
| 169-172 | flow regulators |
| 5300 | continuous workflow heterogeneous |
| | biological sample processing network |
| 5302 | client PC |
| 5304 | client PC |
| 5306 | Nth client PC |
| 5307 | client PC |
| 5308 | system manager SM client software |
| 5309 | Internet |
| 5310 | Lab Information System LIS |
| 5311 | Intelligent Reference Library |
| 5312 | LIS agent |
| 5313 | Service Module |
| 5314 | System manager server SMS |
| 5315 | Network |
| 5316 | Server |
| 5318 | Network |
| 5320 | Stainer |
| 5322 | Stainer |
| 5323 | Distributed Controller |
| 5324 | Nth Stainer |
| 5326 | Slide Imager |
| 5328 | Embedded PC |
| 5330 | Master PCBA |
| 5332 | Drawer Stack PCBA |
| 5334 | Low-level Assembly |
| 5336 | Drawer Assembly |
| 5338 | Hardware |
| 5340 | Automated microtome |
| 5342 | Tissue processor |
| 5344 | Special stainer (non-antibody) |
| 5346 | In-situ hybridization stainer |
| 5348 | Flow cytometry analyzer |
| 5350 | Flow cytometry sorter |
| 5351 | Sample transporter |
| 5352 | Stainer Control Software SCS |
| 5354 | Bridge/Router |

1. A biological sample processing system, comprising:
a biological sample processing apparatus for processing of biological samples arranged on microscope slides;
a production unit connected to the biological sample processing apparatus, the production unit comprising:
a first ingredient source of a first ingredient;
a second ingredient source of a second ingredient;
a mixer station comprising a mixing chamber having a mixer inlet and a mixer outlet, the mixer station being configured to mix ingredients in the mixing chamber to produce a liquid product;
a first supply conduit for supply of an amount of the first ingredient from the first ingredient source to the mixing chamber via the mixer inlet;
a second supply conduit for supply of an amount of the second ingredient from the second ingredient source to the mixing chamber via the mixer inlet;
a delivery conduit for transportation of an amount of the liquid product from the mixing chamber via the mixer outlet; and
a production controller arranged in communication with the production unit, the production controller comprising:
a processor; and
a memory device in communication with the processor, the memory device comprising a computer program which the processor is configured to access to control the operation of the production unit.

2. The biological sample processing system of claim 1, further comprising a sample processing apparatus controller SC arranged in communication with the sample processing apparatus, and being configured to:
control and monitor various processes, functions, or components, that are implemented on or relate to the operation of the sample processing apparatus; and to
schedule the production of liquids in the production unit.

3. The biological sample processing system of claim 2, further comprising a system manager server SMS arranged in communication with the production controller and the sample processing apparatus controller SC, and configured to control the production controller, and wherein the sample processing apparatus controller SC is configured to transfer data to and from the system manager server SMS.

4. The biological sample processing system of claim 3, further comprising a system manager SM arranged in communication with the system manager server SMS and configured to provide a user to connect to, and to transfer data to and from the system manager server SMS.

5. The biological sample processing system of claim 1, further comprising a water tap, water conduit, a water supply regulator for supplying an amount of water to the first ingredient source or to the mixer station, and a purifier arranged to purify the water transported in water conduit, wherein the purifier is a filtration device or a separator configured to use reverse osmosis to purify the water.

6. The biological sample processing system of claim 1, further comprising at least one quality control station arranged to control the quality of liquid supplied to, supplied within or supplied from the production unit.

7. The biological sample processing system of claim 1, further comprising:
a first supply flow regulator arranged at the first supply conduit, arranged to be controlled by the production controller and arranged to supply an amount of the first ingredient to the mixing chamber in response to control instructions from the production controller;
a second supply flow regulator arranged at the second supply conduit, arranged to be controlled by the production controller and arranged to supply an amount of the second ingredient to the mixing chamber in response to control instructions from the production controller; and
a delivery flow regulator arranged at the delivery conduit, arranged to be controlled by the production controller and arranged to transport an amount of the liquid product from the mixing chamber in response to control instructions from the production controller.

8. The biological sample processing system of claim 7, wherein the delivery flow regulator is configured to transport an amount of the liquid product to a second or third quality control station arranged to control the quality of the liquid product, and configured to transport quality controlled liquid product to a production unit outlet or to a liquid storage station for storage.

9. The biological sample processing system of claim 8, further comprising an antimicrobial device arranged in the liquid storage station and configured to prevent bacterial growth in the liquid storage station, and wherein the antimicrobial device comprises a heat source or a UV source.

10. The biological sample processing system of claim 8, further comprising one or more fluid containers comprised on one or more fluidics carts, the one or more fluid containers being in fluid communication with the production unit outlet, whereby liquid product can be supplied from the production unit to one or more of the fluid containers, and wherein the fluidics cart is arranged in communication with the sample processing apparatus controller SC that is configured to control the supply of fluid from the fluid container on the fluidics cart to the sample processing apparatus.

11. The biological sample processing system of claim 1, further comprising determination means for determining the amount of ingredient comprised in the mixing chamber, the determination means being arranged at the mixer station and arranged in communication with the production controller, and being configured to communicate the amount of ingredient to the production controller, whereby the production controller can determine if more ingredient is to be supplied or if sufficient ingredient has been supplied and whereby the production unit controller can control the production unit accordingly.

12. The biological sample processing system of claim 1, wherein at least one of the first and second ingredient sources is configured as a flexible bag comprising the first or second ingredient, and wherein the flexible bag is comprised in a box in a so-called bag-in-a-box configuration.

13. The biological sample processing system of claim 1, wherein the liquid product is produced as a batch or is produced continuously, and/or wherein the liquid product is produced on demand.

14. The biological sample processing system of claim 1, wherein the first ingredient is purified water, the second ingredient is a buffer concentrate and the liquid product is a buffer solution.

15. The biological sample processing system of claim 1, wherein the production controller is configured to control the production unit to produce the liquid product in dependence on the consumption of the liquid product by the sample processing apparatus.

16. A method for liquid production in a biological sample processing system, comprising the steps of:
providing a biological sample processing apparatus for processing of biological samples arranged on microscope slides;
providing a production unit connected to the biological sample processing apparatus, the production unit comprising:
a first ingredient source of a first ingredient;
a second ingredient source of a second ingredient;
a mixer station comprising a mixing chamber having a mixer inlet and a mixer outlet, the mixer station being configured to mix ingredients in the mixing chamber to produce a liquid product;
providing a production controller arranged in communication with the production unit, the production controller comprising:
a processor; and
a memory device in communication with the processor, the memory device comprising a computer program which the processor is configured to access to control the operation of the production unit;
supplying an amount of the first ingredient from the first ingredient source to the mixing chamber in dependence of control instructions from the production controller; supplying an amount of the second ingredient from the second ingredient source to the mixing chamber in dependence of control instructions from the production controller;
mixing in the mixer chamber the amount of the first ingredient and the amount second ingredient in dependence of control instructions from the production controller to produce the liquid product; and
transporting an amount of the liquid product from the mixing chamber via the mixer outlet.

17. The method of claim 16, further comprising the steps of:
providing a sample processing apparatus controller SC in communication with the sample processing apparatus;
controlling and monitoring various processes, functions, or components, that are implemented on or relates to the operation of the sample processing apparatus by means of the sample processing apparatus controller SC; and
scheduling the production of liquids in the production unit by means of the sample processing apparatus controller SC.

18. The method of claim 17, further comprising the steps of:
providing a system manager server SMS arranged in communication with the production controller and the sample processing apparatus controller SC; and
controlling the production controller by means of the system manager server SMS.

19. The method of claim 18, further comprising the steps of:
providing a system manager SM arranged in communication with the system manager server SMS;
providing a user to connect to, and to transfer data to and from the system manager server SMS by means of the a system manager SM.

20. The method of claim 19, further comprising the step of providing at least one quality control station arranged to control the quality of liquid supplied to, supplied within or supplied from the production unit.

21. The method of claim 16, further comprising the steps of:
providing a production unit outlet connected to a liquid product tap;
transporting an amount of the liquid product from the mixing chamber to the production unit outlet; and
withdrawing an amount of the liquid product from the production unit by means of the liquid production tap.

22. The method of claim 16, further comprising the steps of:
providing a production unit outlet connected to one or more fluid containers, and
supplying an amount of the liquid product from the production unit to one or more of the fluid containers.

23. The method of claim 16, further comprising the step of producing the liquid product as a batch or continuously, and/or the step of producing the liquid product on demand.

24. The method of claim 16, further comprising the step of controlling the production unit to produce the liquid product in dependence on the consumption of the liquid product by the sample processing apparatus.

25. (canceled)

26. A computer-readable medium that stores computer program parts, which when executed by a processor controls steps in a method according to claim 16.

27. A computer-readable medium that stores computer program parts, which when executed by a processor controls steps in a method according to claim 17.

28. A computer-readable medium that stores computer program parts, which when executed by a processor controls steps in a method according to claim 18.

29. A computer-readable medium that stores computer program parts, which when executed by a processor controls steps in a method according to claim 19.

30. A computer-readable medium that stores computer program parts, which when executed by a processor controls steps in a method according to claim 20.

31. A computer-readable medium that stores computer program parts, which when executed by a processor controls steps in a method according to claim 21.

32. A computer-readable medium that stores computer program parts, which when executed by a processor controls steps in a method according to claim 22.

33. A computer-readable medium that stores computer program parts, which when executed by a processor controls steps in a method according to claim 23.

34. A computer-readable medium that stores computer program parts, which when executed by a processor controls steps in a method according to claim 24.
